(19) United States
(12) Patent Application Publication
(10) Pub. No.: US 2006/0286624 A1
(43) Pub. Date: Dec. 21, 2006

(54) Method for identifying selective growth inhibitors

(76) Inventor: Michel J. Hamelin, Jersey City, NJ (US)

Correspondence Address: MERCK AND CO., INC, P O BOX 2000, RAHWAY, NJ, 07065-0907, US

(21) Appl. No.: 10/569,622

(22) PCT Filed: Aug. 20, 2004
(86) PCT No.: PCT/US04/27210

§ 371 Date: Feb. 24, 2006

Related U.S. Application Data
(60) Provisional application No. 60/497,750, filed on Aug. 26, 2003.

(51) Int. Cl. C12Q 1/18

(52) U.S. Cl. 435/32

(57) ABSTRACT

Selective growth inhibitors are identified by (i) providing a semi-solid nutrient medium (e.g., an agar plate) co-seeded with a strain of cells (e.g., bacteria or fungi) that is sensitive to growth inhibitors having a particular mode of action and with a strain that is essentially the same as the sensitive strain except that it is resistant to said growth inhibitors, wherein the sensitive strain is co-seeded in the nutrient medium in an amount substantially in excess of that of the resistant strain; (ii) treating the medium with a test substance; (iii) incubating the treated medium under conditions suitable for the growth of both the sensitive and the resistant strains; and (iv) examining the incubated treated medium for growth inhibition. The observation in (iv) of a zone of no growth except for isolated colonies of the resistant strain is an indication that the test substance is a selective growth inhibitor having the mode of action of interest.

This application claims the benefit of U.S. Provisional Application No. 60/497,750, filed Aug. 26, 2003, the disclosure of which is herein incorporated by reference in its entirety.

### FIELD OF THE INVENTION

The invention relates to the identification of substances that inhibit the growth of cells (e.g., bacteria or fungi) by acting against a product of the cell (e.g., an enzyme) whose proper functioning is required for cell growth or survival. More particularly, the present invention is directed to a semi-solid-medium based, whole-cell assay for selectively screening for antimicrobial compounds with particular mechanisms of action. In the present invention, the semi-solid medium is co-seeded with two strains of cells wherein the two strains are substantially the same except that one strain is sensitive and the other is resistant to antigrowth agents with a particular mechanism of action and wherein the sensitive strain is co-seeded in an amount substantially in excess of the resistant strain. The co-seeded medium is incubated in the presence of a test substance under conditions suitable for growth of the strains, followed by post-growth examination for the appearance and nature of growth inhibition zones in the semi-solid medium in order to determine whether the test substance is a growth inhibitor with the particular mechanism of action.

### BACKGROUND OF THE INVENTION

Pathogenic strains of bacteria which represent a major threat to public health include Staphylococcus, Streptococcus, Enterococcus, Escherichia, Klebsiella, Haemophilus, Enterobacter, Acinetobacter, Bacillus, Stenotrophomonas, Salmonella, Burkholderia, and Pseudomonas, specifically including the strains Streptococcus pneumonia, Staphylococcus aureus, Enterococcus faecalis, Enterococcus faecium, Escherichia coli, Klebsiella pneumoniae, Haemophilus influenzae, Enterobacter cloacae, Pseudomonas aeruginosa, Acinetobacter baumanii, Bacillus subtilis, Stenotrophomonas maltophilia, Salmonella typhimurium, and Burkholderia cepacia. Pathogenic bacteria cause such diseases as pneumonia, typhoid, diarrhea, and tuberculosis. Antibiotic agents have been developed to combat such diseases, including, for example, amikacin, gentamicin, tobramycin, amoxicillin, amphotericin B, ampicillin, atovaquone, azithromycin, cefazolin, cefepime, cefotaxime, cefotetan, cefpodoxime, ceftazidime, ceftizoxime, ceftriaxone, cefuroxime, cephalexin, chloramphenicol, clotrimazole, ciprofloxacin, clarithromycin, clindamycin, dicloxacillin, doxycycline, erthromycin lactobionate, imipenem, izoniazid, kanamycin, linezolid, metronidazole, nafcillin, nitrofurantoin, nystatin, penicillin, pentamidine, piperacillin, rifampin, ticarcillin, trimethoprim, and vancomycin.

While these agents are effective against pathogenic bacteria and are thus useful in the treatment of disease conditions associated with the presence of bacteria, many of the clinically important strains of bacteria are becoming resistant to the known antibiotics. For example, Enterococci that are resistant to a vast array of antimicrobial drugs, including cell wall active agents, aminoglycosides, penicillin, ampicillin, and vancomycin, have been observed. Staphylococcus aureus, the most frequent causative agent of infections in the hospital environment, is resistant to many of the known antibiotics, making it difficult to treat such infections. Further description of the nature of antibiotics and the development of antibiotic resistance is found in Walsh, Nature 2000, 406: 775-781. Accordingly, there is a continuing need to discover new antibacterial agents with novel mechanisms of action, and thus a low probability for preselected resistance in the clinic, to combat the rise of resistant strains in both the hospital and community environments. There is a concomitant need to develop reliable, high-throughput methods for identifying new antibacterials having a specific mechanism of action and bacterial cell wall permeability.

Pathogenic strains of fungi which represent a major threat to public health include: Cryptococcus spp., Candida spp., Aspergillus spp., Histoplasma spp., Coccidioides spp., Paracoccidioides spp., Blastomyces spp., Fusarium spp., Sporothrix spp., Trichosporon spp., Scedosporium, Rhizopus spp., Pseudallescheria spp., dermatophytes, Paeciliomyces spp., Alternaria spp., Curvularia spp., Exophiala spp., Schizosaccharomyces spp., Wangiella spp., Dematiaceous fungi and Pneumocystis spp., and specifically Saccharomyces cerevisiae, Aspergillus nidulans, Aspergillus fumigatus, Cryptococcus neoformans, Coccidioides immitis, Schizosaccharomyces pombe, Pneumocystis carinii, and Candida albicans. Current therapies to these fungi are inadequate due to a poor spectrum of activity and toxicity. Resistance to antifungals is not as prevalent as found with antibacterial agents, but there is a steady increase in resistance to antifungal agents used in a hospital setting. The immuno-compromised patient is particularly susceptible to fungal infections where drug interactions with existing antifungal therapies can be an issue. There is a need for new antifungals with novel mechanisms of action that exhibit improved therapeutic profiles.

The standard approach for the discovery of antibacterials is to expose a test compound or mixture of compounds to the bacterial cells, and measure the level of cellular growth inhibition. Growth inhibition or cell death is typically determined in a liquid medium (i.e., the “liquid growth assay”) by measuring the optical density of the culture following an 18-hour growth period. The growth inhibition of a test compound is typically reported in terms of minimum inhibitory concentration (MIC) required to inhibit 99.9 percent of the cell growth or where no visible growth is observed. Such assays are described, for example, in W. Hewitt, Microbiology Assay (Academic Press, New York, N.Y., 1977), pp. 1-261. Another document describing the determination of MICs is “Methods for Dilution Antimicrobial Susceptibility Tests for Bacteria that Grow Aerobically”, NCCLS Document M7-A4, National Committee for Clinical Laboratory Standards, Wayne, Pa., 4^{th} edition, 1997. When liquid culture-based assays are performed on a single concentration of compound, they are economical, amenable to high-throughput screening (HTS) and suitable for screening of compounds soluble in low levels of solvent (e.g., 0.2% DMSO). The disadvantages of HTS liquid cell growth assays are the limited capability to detect water-insoluble compounds, the difficulty in determining a compound's relative potency, the repeated identification of commonly known antibacterials (i.e., high rate of redundancy), and a predisposition to identify compounds which are potent but also toxic to mammalian cells.

A semi-solid medium approach for discovering bacterial growth inhibitors is the agar zone of inhibition assay (or “agar assay”), wherein agar in a dish or plate is impregnated with bacteria and the compound is applied to the plate either on a paper disk, in a precast well, or as a drop on the agar surface. Bacterial growth inhibition or cell death is typically determined by measuring zones of inhibition in the assay plate. The conventional agar assay (or, more generally, the semi-solid medium-based assay) is further described, for example, in J. F. Acar and F. W. Goldstein, “Procedure for testing antimicrobial agents in agar media: theoretical considerations” in: Antibiotics in Laboratory Medicine, edited by V. Lorian (Williams & Wilkins, Baltimore Md., 1996), pp 1-51. Semi-solid medium-based assays are well-suited for solid samples and fresh natural products and extracts of natural products. Difficulties in the rapid and accurate detection and measurement of inhibition zones restrict the use of semi-solid-medium based assays for high-throughput screening. As with the conventional liquid assay, a limitation of these screens is the high rate of redundancy in the mode of action of identified active agents; e.g., compounds acting at cell wall components are found with a high frequency.

Biochemical, in vitro-based assays can also be used for antibacterial discovery. An advantage of this approach is that it permits the rapid identification of substances acting at a very specific (and predetermined) target. A major disadvantage of this mechanism-based approach has been the identification of compounds that exhibit potent biochemical inhibitory activity but lack antibacterial activity because of instability, poor cell membrane permeability, or the like.

Selective target screening of antimicrobial substances can be performed using microbial strains with altered sensitivities to certain classes of compounds (e.g., antibacterials with a particular mechanism of action). Drug-resistant and -sensitive strains can be tested in liquid or semi-solid-media and the differential sensitivities measured. This approach uses a mutated strain to screen for compounds that act selectively and have antimicrobial activity. For example, to screen for antibiotics that act like tetracyclin, a tetracyclin-sensitive strain can be compared to a resistant strain wherein any compound affecting the sensitive strain but not the resistant one is singled out as positive. Approaches for genetically engineering bacterial strains with altered sensitivities are outlined in Ji et al., Science 2001, 293: 2266-2269, Forsyth et al., Mol. Microbiol. 2002, 43: 1387-1400, and DeVito et al., Nat. Biotechnol. 2002, 20: 478-483). The screening for antimicrobials (e.g., antibacterials) on a drug resistant mutant microbe will allow the identification of antimicrobials that act at that target, but should miss antimicrobials that act at other targets. These screens are typically performed in liquid (comparing two vials or wells—one containing the sensitive cells and the other containing the resistant cells) or on two plates. Drawbacks for the liquid format include the solubility issues mentioned above, plus the need to perform the screen at a drug concentration that will selectively affect the sensitive strain but not the resistant one. The semi-solid two-plate format, because of the radial diffusion of experimental samples from higher to lower concentrations, allows for a greater sample concentration range at which screening can be performed. Drawbacks of the semi-solid two-plate format include the duplication of effort and material, time-consuming zone measurements, error due to variation in the experimental handling of the two plates, and discrepancies related to screening compounds with different diffusion properties. These comparative assays are also unsuitable for screening natural product samples which are very difficult to obtain in duplicate.

An alternative to using semi-solid two-plate assays are single plate assays in which both strains are co-seeded in roughly equal amounts onto the same plate. In this case, one of the two strains (usually the resistant one) has been genetically modified with a reporter system (e.g., a green fluorescent protein such the one described in Chalfie et al., Science 1994, 263: 802-805) in order to discriminate it from the reference strain. Drawbacks of these reporter-based systems include artifactual results and the requirement for genetic manipulation of one of the strains.

An alternative approach using a single-plate assay has recently been developed wherein a “sensitized” bacterial strain is seeded onto an agar plate, and zones of inhibition are generated by treating the medium with antimicrobial substances. Substances that act selectively at the target for which the strain has been sensitized (i.e., selective growth inhibitors) are distinguished from the non-selective ones by the presence of a few small resistant colonies within the selective zones. The “selectively resistant” colonies result from low level, spontaneous generation of “revertant” cells from the sensitized ones. Two requirements for using such a single-plate assay are that 1) a strain be genetically engineered to become “sensitive” and 2) that the genetic element responsible for the increased sensitivity be relatively unstable. Because of these requirements, the assay is of limited potential use. The assay has been employed using antisense-sensitized bacterial strains; e.g., recombinant bacterial cells that (i) contain a nucleic acid fragment encoding anti-sense RNA (asRNA) wherein expression of the asRNA pre-sensitizes the cells to substances acting at a particular gene product (e.g., a protein or RNA) and (ii) are unstable in that they can lose the capability to express the asRNA under certain growth conditions, thereby resulting in the appearance of revertant cells in the presence of selective growth inhibitors. This assay is further described in WO 2004/009835, which is the publication of International Application No. PCT/US03/21726, entitled “Method for Identifying Cellular Growth Inhibitors”, filed Jul. 14, 2003. Forsyth et. al. (Mol. Microbiol. 2002, 43: 1387-1400) discloses antisense-sensitized bacterial strains suitable for use in the assay.

While much of the discussion in this section has focused on known methods for discovering antibacterials, the same or similar methods have been employed for the discovery of antifungals, methods that suffer from the same or similar disadvantages.

There is a need for the development of new and/or improved antimicrobial screening methods that minimize or avoid drawbacks associated with the assays described above.

### SUMMARY OF THE INVENTION

The present invention is a method which permits the selective screening for growth-inhibiting substances (e.g., single compounds or natural products) having a known mechanism of action; i.e., substances which inhibit or otherwise adversely interfere with the function of a cellular product that is required for the growth or survival of the cell (e.g., a bacterial or fungal cell). As used herein, the term “inhibiting” (or “inhibition”) in reference to cellular growth means the reduction or suppression of growth of the cells (e.g., bacterial cells). More particularly, the present invention includes a method for identifying selective growth inhibitors, which comprises:

(A) providing a semi-solid nutrient medium (e.g., an agar plate) co-seeded with a first and a second strain of cells; wherein (i) the two strains are substantially the same except that the growth of the first strain (the “sensitive” strain) is sensitive to exposure to an inhibitor that acts by selectively inhibiting the function of a product of the cell required for its growth or survival, and the growth of the second strain (the “resistant” strain) is substantially resistant to exposure to the selective inhibitor, and (ii) first strain is present in the nutrient medium in an amount substantially in excess of the second strain;

(B) treating the nutrient medium with a test substance;

(C) incubating the treated medium under conditions suitable for growth of the two strains of cells; and

(D) examining the incubated nutrient medium for growth inhibition; wherein:
(1) if the incubated nutrient medium exhibits a clear zone indicative of essentially no growth due to the death of all or substantially all of the cells, then the test substance is or contains a growth inhibitor that does not selectively inhibit the targeted cell product;
(2) if the incubated nutrient medium does not exhibit a zone of no growth due to the survival and growth of all or substantially all of the cells, then the test substance is not or does not contain a growth inhibitor; and
(3) if the incubated nutrient medium exhibits a zone of no growth except for one or more small cell colonies in the zone due to the survival and growth of the second strain, then the test substance is or contains a growth inhibitor that selectively inhibits the targeted cell product.

A key feature of the present invention is the unequal co-seeding of the nutrient medium with the sensitive and resistant strains. Stated alternatively, the medium is seeded with a sensitive strain that is “spiked” with a relatively small amount of the resistant strain. Following treatment of the co-seeded medium (e.g., an assay plate) with the test substance and incubation of the treated medium, the nature of the test substance can be determined from inspection of the incubated medium, wherein a selective growth inhibitor will generate a zone of no growth punctuated with colonies of the resistant strain. The observation of the isolated surviving resistant colonies indicates that the screened test substance is interacting with the targeted pathway (e.g., an enzyme or gene product) by which the two strains differ. Absent the unequal co-seeding of the strains, the growth of isolated resistant colonies in this circumstance would not be observed and selective growth inhibitors could not be identified. More particularly, the unequal co-seeding results in a relative scarcity of the resistant strain (e.g., resistant bacteria) in the treated, incubated medium. Individual colonies of the resistant and sensitive strains will grow to a size that is roughly proportional to the distance between them. In the zone of no growth of the sensitive strain, the individual colonies of the resistant strain grow because their growth is not being repressed by the test substance (v. colonies of the sensitive strain) and will grow to a visually observable size because the relatively sparse resistant colonies will grow to a much larger diameter than they would when crowded by growth of colonies of the sensitive strain.

The method of the present invention has advantages over the assays described above in the Background of the Invention. In contrast to the liquid growth assay, the method of the invention does not require titration, can detect and measure the activity of water insoluble compounds, and eliminates the identification of generally toxic compounds. Unlike the standard agar assay, the present invention does not involve the measurement of zone diameters. Furthermore, the method of the present invention identifies substances with specific mechanisms of action, whereas the liquid growth and standard agar assays typically do not (unless two strains are compared, which would double the effort). Although biochemical assays identify compounds with specific biochemical inhibitory activity, these assays do not, in contrast to the whole-cell method of the invention, necessarily identify compounds having antimicrobial activity. The present invention also avoids the drawbacks characteristic of the two-plate assay system, including the duplication of effort and material due to the use of two plates instead of one, the zone measurements, and the potential experimental error introduced by differences in the handling of each plate in the pair. The method of the present invention also avoids the need for the genetic engineering of one strain characteristic of the reporter-based systems and the antisense assays. Further in contrast to the antisense assays, the method of the present invention is not restricted to the identification of compounds acting at a specific gene product. Overall, the method of the present invention is efficient, specific, sensitive, and simple.

Various other embodiments, aspects and features of the present invention are either further described in or will be apparent from the ensuing description, examples and appended claims.

### BRIEF DESCRIPTION OF THE DRAWINGS

FIG. 1 shows the growth inhibition zones obtained for the test substances (A) chloramphenicol, (B) streptomycin, and (C) cefoxitin on assay plates resulting from the assay set forth in Example 1, Runs 1 to 3 respectively.

FIG. 2 shows the growth inhibition zones for (A) ciprofloxacin and (B) chloramphenicol on assay plates resulting from the assay set forth in Example 1, Runs 4 and 5 respectively.

FIG. 3 shows the growth inhibition zone for ampicillin on the assay plate resulting from the assay set forth in Example 1, Run 6.

FIG. 4 shows the growth inhibition zones for (A) tetracyclin and (B) streptomycin on the assay plate resulting from the assay set forth in Example 1, Runs 7 and 8 respectively.

FIG. 5 shows the growth inhibition zones for (A) enfumafungin and (B) sordarin on the assay plate resulting from the assay set forth in Example 2, Runs 9 and 10 respectively.

### DETAILED DESCRIPTION OF THE INVENTION

The present invention includes the method comprising Steps A to D as set forth above under the Summary of the Invention. Step A of the present invention provides a semi-solid nutrient medium co-seeded with a first (sensitive) strain and a second (resistant) strain of cells, wherein the first strain is present in an amount substantially in excess of the second strain. The cell strains are suitably prokaryotes or eukaryotes, and are especially bacteria or fungi. The term “substantially in excess” means that at least about 95% (preferably at least about 96%, more preferably at least about 99%) of the cells in the nutrient medium are cells from the sensitive strain. In one embodiment, the first and second strains are co-seeded in a ratio of at least about 25 cells of the first strain per cell of the second strain (alternatively referred to as a co-seeding ratio of at least about 25 to 1). In another embodiment, the co-seeding ratio is at least about 100:1 (i.e., at least about 100 cells of the sensitive strain per cell of the resistant strain). In still another embodiment, the co-seeding ratio is at least about 500:1. When the co-seeded medium is incubated under conditions suitable for the growth of both strains (described below) but in the presence of a substance that inhibits the growth of the sensitive strain but with little or no effect on the growth of the resistant strain, only growth of the resistant strain will be evident. The density of the resistant cells in the medium is relatively low as a result of originally co-seeding the medium with a substantial excess of sensitive cells, and thus the growth in the presence of a selective growth inhibitor will typically be seen as isolated, individual colonies.

At low co-seeding ratios (i.e., below about 25:1), the resistant colonies can become relatively small in diameter and high in density, as a result of which the growth zone in the co-seeded medium in the presence of a selective growth inhibitor can be visually hazy or cloudy and thus difficult to distinguish from the growth zone that would be obtained from growth in the presence of a non-growth inhibiting test substance. At high co-seeding ratios (i.e., above about 5000:1), the density of resistant colonies can be so low that their occurrence can be difficult to distinguish from the occurrence of colonies arising from random events, such as colonies due to contamination or due to spontaneous mutants arising from the sensitive strain. Accordingly, in another embodiment of the present invention, the first strain and the second strain are co-seeded in the semi-solid nutrient medium in a ratio in a range of from about 25:1 to about 5000:1 (e.g., from about 25:1 to about 1000:1). In another embodiment, the co-seeding ratio is in a range of from about 25:1 to about 2500:1 (e.g., from about 25:1 to about 500:1). In still another embodiment, the co-seeding ratio is in a range of from about 50:1 to about 1000:1 (e.g., about 100:1). In yet another embodiment, the co-seeding ratio is in a range of from about 500:1 to about 5000:1 (e.g., from about 500:1 to about 1000:1).

Co-seeding of the semi-solid nutrient medium can be carried out as follows: The sensitive and resistant cell strains are each grown overnight in respectively suitable liquid growth media held at a suitable incubation temperature (e.g., about 37° C.) and optionally with continuous agitation. Following growth, the sensitive strain is diluted with the growth medium per se to a suitable concentration of cells. The cell concentration is typically measured in terms of the optical density (OD) or transmittance of the medium at a specified wavelength (e.g., dilution to an optical density in a range of from about 0.01 to about 0.5 at λ=600 nm) relative to a control with a known cell concentration (e.g., the control can be the growth medium per se which contains no cells). The resistant medium is also diluted in an analogous manner, but to a greater extent in order to provide a co-seeding ratio suitable for use in the method of the present invention (e.g., a ratio of sensitive cells to resistant cells of at least about 25:1). Equal portions of the diluted sensitive and resistant cells are then mixed into the molten nutrient medium (e.g., molten agar) and the mixture poured into glass or plastic dishes to form plates. Prior to hardening into the semi-solid form employed in the invention, wells can be cast into the poured, molten mixture. The concentration of cells in the culture following overnight growth and prior to dilution is typically of the order of about 10⁹ cells per mL, as determined by OD measurements. The concentration of sensitive cells in the diluted co-seed mixture used for inoculating the semi-solid medium is typically of the order of from about 10² to about 10⁷ cells per mL. The inoculating concentration will depend upon such factors as the degree of dilution employed and the choice of microbial species. The concentration of resistant cells in the inoculated semi-solid medium will vary in accordance with the sensitive cell concentration such that the ratio of resistant to sensitive cells is suitable for use in the present invention (e.g., the ratio is from about 25:1 to about 5000:1).

As noted in the preceding paragraph, batches of the sensitive and resistant strains of cells suitable for dilution and co-seeding can be obtained by growing a seed sample in a growth medium (which can alternatively referred to as a cultivation medium or an incubation medium) at a suitable temperature. Suitable media for cultivating various strains of cells are well known in the art. Suitable media typically comprise either or both a carbon source (e.g., a sugar such as glucose, xylose or the like) and a nitrogen source (e.g., yeast extract), and optionally other components such as an inorganic salt or a defoaming agent, all dissolved or suspended in water. Among the growth media which can typically be employed are commercially available media such as Luria Bertani (LB) medium (available from Life Technologies, Rockville, Md.), Sabouraud Dextrose (SD) medium (available from Difco, Detroit, Mich.), and Yeast medium (YM) broth (available from Difco). Other defined or synthetic growth media may also be used and the appropriate medium for growth of a particular microorganism will be known by the person of ordinary skill in the art of microbiology or fermentation science. Suitable growth media include those disclosed in J. F. Acar and F. W. Goldstein, “Procedure for testing antimicrobial agents in agar media: theoretical considerations” in: Antibiotics in Laboratory Medicine V. Lorian Ed. (Williams & Wilkins, Baltimore Md., 1996), and in F. Sherman, G. Fink, and J. Hicks, J., Methods in Yeast Genetics, (Cold Spring Harbor Laboratory Press. Cold Spring Harbor, N.Y., 1981).

In Step B of the method of the present invention, the co-seeded nutrient medium is treated with the test substance. The nutrient medium can suitably be treated with the test substance by bringing the substance into contact with the surface of the medium. In one embodiment, treating comprises applying the test substance (neat, dissolved in a suitable solvent, or dispersed in a suitable fluid medium) to the surface of the nutrient medium, such as by placing (also referred to as “spotting” or “dot spotting”) a drop of liquid containing the test substance directly onto the surface of the nutrient medium (e.g., agar), by placing the liquid containing the substance in a well molded into the semi-solid medium, or by bringing a piece of filter paper (e.g., a paper disc) having the test substance absorbed thereon into contact with the surface of the nutrient medium. Alternatively, a solid sample (e.g., an agar strip on which microorganisms have been grown with the concomitant production of natural products) containing the test substance(s) can be placed directly on the surface of the nutrient medium. Regardless of the mode of application, the test substance(s) diffuse from the point of contact into the nutrient medium.

The concentration and total amount of the test substance applied to the nutrient medium is not critical, because the semi-solid nutrient medium (e.g., agar) allows the gradual diffusion of the substance from a comparatively high concentration (at the application site) to a comparatively low concentration (at the edge of the test zone), thereby permitting the screening for active drugs over a wide range of potency. Thus, suitable concentrations of test substances include, but are not limited to, those in a range of from about 1 ρM to about 5 M, and typical concentrations are those in a range of from about 1 nM to 100 μM. The nutrient medium is typically treated in a given test with about 0.005 to 0.1 mL of the test solution.

When an agar strip or the like containing a growing colony of fungi or bacteria is applied to the nutrient medium, the concentration of the test substance(s) diffusing therefrom is typically not known and/or not easily controlled, but, because the concentration is not critical for the reason given above, such strips can be screened/tested via the method of present invention without significant difficulty.

The incubation in Step C can suitably be conducted under any conditions that permit the growth of the first and second strains of cells and the development of a growth inhibition zone. The incubation is suitably conducted at a temperature in a range of from about 20° C. to about 45° C. (e.g., from about 25° C. to about 45° C.), and is typically conducted at a temperature in a range of from about 30° C. to about 42° C. In one embodiment, the incubation temperature is in a range of from about 35° C. to about 39° C. (e.g., about 37° C.). The incubation time can vary widely depending upon, inter alia, the choice of nutrient medium, the incubation temperature, and the identity of the cell strains (e.g., prokaryotic v. eukaryotic). Nonetheless, the incubation is typically conducted for a time in a range of from about 4 to about 120 hours (e.g., from about 8 to about 120 hours, or from about 4 to about 96 hours, or from about 8 to about 96 hours). In one embodiment, the cell strains are either strains of bacteria or strains of yeast and the incubation time is in a range of from about 8 to about 48 hours (e.g., from about 16 to about 36 hours).

The examination in Step D is typically a visual examination of the incubated test area for the existence of a growth inhibition zone. If a clear inhibition zone is observed, then the test substance is or contains a non-selective growth inhibitor. If an inhibition zone is observed that is clear except for the presence of small cell colonies, then the test substance is or contains a selective growth inhibitor. If a clear zone is absent (i.e., no indication of the death of any cells), then the test substance is not or does not contain a growth inhibitor.

The examination in Step D of the present invention can in extreme instances lead to false negatives; i.e., if a test substance is in fact a very potent selective inhibitor and the substance is applied at too high a concentration, “selective” concentrations will not be obtained within the diffusion boundary of the inhibition zone (which typically have a diameter of from about 1 to about 10 cm). Thus, the test substance will kill both the sensitive and resistant strains which will result in the exhibition of a clear zone of essentially no growth and will lead to the erroneous conclusion that the test substance is not selective. If a false negative is suspected (e.g., substances structurally similar to the test substance have exhibited potent and selective inhibition), the screen can be repeated at lower concentrations as a check on the initial result. On the other hand, it is emphasized that, when the substances are tested within a reasonable concentration range (e.g., from about 1 nM to about 100 μM), the occurrence of false negatives with the method of the present invention should be rare.

In accordance with the foregoing discussion, embodiments of the present invention include the method as originally set forth above in the Summary of the Invention incorporating any one or more of the following features:

(a) the semi-solid nutrient medium is an agar plate or an agarose plate;

(b) treating in Step B comprises applying the test substance to the surface of the semi-solid nutrient medium;

(c) one of:

(c1) the incubation in Step C is conducted at a temperature in a range of from about 20° C. to about 45° C.;

(c2) the incubation in Step C is conducted at a temperature in a range of from about 25° C. to about 45° C.;

(c3) the incubation in Step C is conducted at a temperature in a range of from about 30° C. to about 42° C.; or

(c4) the incubation in Step C is conducted at a temperature in a range of from about 35° C. to about 39° C. (e.g., about 37° C.);

(d) one of:

(d1) the incubation in Step C is conducted for a time in a range of from about 4 to about 120 hours;

(d2) the incubation in Step C is conducted for a time in a range of from about 4 to about 96 hours;

(d3) the incubation in Step C is conducted for a time in a range of from about 8 to about 120 hours;

(d4) the incubation in Step C is conducted for a time in a range of from about 8 to about 96 hours;

(d5) the incubation in Step C is conducted for a time in a range of from about 8 to about 48 hours; or

(d6) the incubation in Step C is conducted for a time in a range of from about 16 to about 36 hours;

(e) one of:

(e1) the first strain and the second strain are co-seeded in the semi-solid nutrient medium in a ratio of at least about 25 cells of the first strain per cell of the second strain;

(e2) the first strain and the second strain are co-seeded in the semi-solid nutrient medium in a ratio of at least about 100 cells of the first strain per cell of the second strain;

(e3) the first strain and the second strain are co-seeded in the semi-solid nutrient medium in a ratio of at least about 500 cells of the first strain per cell of the second strain;

(e4) the first strain and the second strain are co-seeded in the semi-solid nutrient medium in a ratio in a range of from about 25 to about 5000 cells of the first strain per cell of the second strain;

(e5) the first strain and the second strain are co-seeded in the semi-solid nutrient medium in a ratio in a range of from about 25 to about 2500 cells of the first strain per cell of the second strain;

(e6) the first strain and the second strain are co-seeded in the semi-solid nutrient medium in a ratio in a range of from about 25 to about 1000 cells of the first strain per cell of the second strain; or

(e7) the first strain and the second strain are co-seeded in the semi-solid nutrient medium in a ratio in a range of from about 50 to about 1000 cells of the first strain per cell of the second strain;

(f) in Step B, the nutrient medium is treated with a plurality of test substances, with each test substance being applied to a distinct portion of the surface of the nutrient medium (the term “distinct” here means that each test substance is applied to a different portion of the medium's surface such that the resulting growth/growth inhibition zone is separate from and not overlapping with the zone for any other test substance); and wherein in Step D the nutrient medium is examined for growth inhibition by each of the plurality of test substances by examining each of the corresponding distinct portions of the nutrient surface;

(g) the growth of the first strain is repressed by exposure to an inhibitor that acts by selectively inhibiting the function of a specific gene product of the cell required for the cell's growth or survival, and the growth of the second strain is substantially resistant to exposure to the selective inhibitor; and

(h) one of:

(h1) the first and second strains are either bacterial strains or fungal strains;

(h2) the first and second strains of cells are bacterial strains and the test substance is being tested to determine whether it is an antibacterial agent that acts by selectively inhibiting the function of a cell product required for the growth or survival of the bacteria;

(h3) the feature as described in (h2), wherein the bacterial strains are selected from the group consisting of Staphylococcus, Streptococcus, Enterococcus, Escherichia, Klebsiella, Haemophilus, Enterobacter, Acinetobacter, Bacillus, Stenotrophomonas, Burkholderia, Salmonella, and Pseudomonas;

(h4) the feature as described in (h2), wherein the bacterial strains are selected from the group consisting of Streptococcus pneumonia, Staphylococcus aureus, Enterococcus faecalis, Enterococcus faecium, Escherichia coli, Klebsiella pneumoniae, Haemophilus influenzae, Enterobacter cloacae, Pseudomonas aeruginosa, Acinetobacter baumanii, Bacillus subtilis, Stenotrophomonas maltophilia, Salmonella typhimurium, and Burkholderia cepacia;

(h5) the first and second strains of cells are fungal strains and the test substance is being tested to determine whether it is an antifungal agent that acts by selectively inhibiting the function of a cell product required for the growth or survival of the fungi;

(h6) the feature as described in (h5), wherein the fungal strains are selected from the group consisting of Cryptococcus spp., Candida spp., Aspergillus spp., Histoplasma spp., Coccidioides spp., Paracoccidioides spp., Blastomyces spp., Fusarium spp., Sporothrix spp., Trichosporon spp., Scedosporium, Rhizopus spp., Pseudallescheria spp., dermatophytes, Paeciliomyces spp., Alternaria spp., Curvularia spp., Exophiala spp., Schizosaccharomyces spp., Wangiella spp., Dematiaceous fungi, Pneumocystis spp, Absidia spp., Blastocystis spp., and Epidermophyton spp.; or

(h7) the feature as described in (h5), wherein the fungal strains are selected from the group consisting of Saccharomyces cerevisiae, Aspergillus nidulans, Aspergillus fumigatus, Cryptococcus neoformans, Coccidioides immitis, Schizosaccharomyces pombe, Pneumocystis carinii, Candida albicans, Absidia corymbifera, Aspergillus flavus, Blastocystis hominis, Blastomyces dermatitidis, Coccidioides immitis, Cryptococcus neoformans, Epidermophyton floccosum, Sporothrix schenckii, and Trichophyton rubrum.

Another embodiment of the present invention is a method for identifying selective growth inhibitors which comprises:

(A) providing an agar plate or an agarose plate, the plate co-seeded with a first and a second strain of cells in a ratio of at least about 25 cells of the first strain per cell of the second strain; wherein the two strains are substantially the same except that the growth of the first strain is sensitive to exposure to an inhibitor that acts by selectively inhibiting the function of a product of the cell required for its growth or survival, and the growth of the second strain is resistant to exposure to the selective inhibitor;

(B) treating the plate with a test substance by applying the test substance to the surface of the plate;

(C) incubating the treated plate at a temperature in a range of from about 20° C. to about 45° C. and for a time in a range of from about 4 to about 96 hours; and

(D) examining the incubated plate for growth inhibition; wherein:
(1) if the incubated plate exhibits a clear zone indicative of essentially no growth due to the death of all or substantially all of the cells, then the test substance is or contains a growth inhibitor that does not selectively inhibit the targeted cell product;
(2) if the incubated plate does not exhibit a zone of no growth due to the survival and growth of all or substantially all of the cells, then the test substance is not or does not contain a growth inhibitor; and
(3) if the incubated plate exhibits a zone of no growth except for one or more small cell colonies in the zone due to the survival and growth of the second strain, then the test substance is or contains a selective growth inhibitor that selectively inhibits the targeted cell product.

Aspects of this embodiment include the process as just described incorporating one or more of the features (c2) or (c3) or (c4), (d4) or (d5) or (d6), (e2) or (e3) or (e4) or (e5) or (e6) or (e7), (f), (g), and (h) as set forth above.

The terms “test substance” and “substance” are used interchangeably and refer to a compound, a mixture of compounds (i.e., at least two compounds), or a natural product sample containing one or more compounds. Thus, the analysis of the cell growth of a test substance may encompass the inhibitory activity of more than one growth inhibitor, including combinations of selective and non-selective inhibitors. The method of the present invention is employed to identify selective growth inhibitors. When a test substance is a single compound and is identified as a selective growth inhibitor in the method of the present invention (i.e., the analysis of the cell growth in Step D of the method of invention determines that the growth falls into category (3)—a clear zone of no growth except for colonies of resistant cells), then the test substance “is” a selective growth inhibitor. When a test substance is a mixture of compounds (e.g., a natural product sample) and is identified as a selective growth inhibitor in the method of the present invention (i.e., the analysis of the cell growth in Step D of the method of invention determines that the growth falls into category (3)), then the test substance “contains” a selective growth inhibitor. Of course, it is to be understood that a selective growth inhibitor, when present as part of a mixture, may not be detectable if one of the other components of the mixture is a non-selective growth inhibitor present at a sufficiently high concentration such that it masks the presence of the selective inhibitor (i.e., a false negative).

Of course, isolated single compounds will normally be tested separately in the method of the present invention, since mixing them together could complicate the interpretation of the results in Step D. Mixtures of compounds are typically tested as mixtures only when obtained or available in that form (e.g., a natural product sample) and it is difficult and/or expensive to separate and isolate the active components therefrom.

The term “semi-solid nutrient medium” is alternatively referred to herein as simply the “nutrient medium”.

As used herein, a “selective growth inhibitor” is an inhibitor which retards or suppresses cellular growth via a specific mechanism of action (also referred to herein as a specific mode of action). More particularly, the growth inhibitor acts by selectively inhibiting a function of a product of the cell that is required for the cell's growth or survival. The method of the present invention can be employed to identify selective growth inhibitors irrespective of their mode of action; i.e., the present invention is not restricted to the identification/screening of any particular type of selective inhibitor. Thus, for example, the present invention can be employed to screen for growth inhibitors that act by selectively inhibiting the function of a specific gene product required for the cell's growth or survival (e.g., an enzyme necessary for cell viability). As another example, the present invention can be employed to identify growth inhibitors that target cell structures that result indirectly from one or more gene products (e.g., the peptidoglycan cell wall which is built by and in part made from proteins) while the gene products themselves are not targeted. As still another example, the method of the present invention can screen for inhibitors that target a structure consisting of several gene products (e.g., a ribosomal subunit).

From the preceding paragraph, it is clear that a “cell product” whose function is required for the growth or survival of the cell refers to the product of a specific gene, or the product resulting from the concerted action of two or more genes, or a cell product formed or obtained from the action or function of one or more gene products. In other words, the cell product can be any component of a cell whose function is required for the cell's growth or survival. In bacteria, the cell product can be, for example, the product of a gene (or genes): controlling multidrug efflux (e.g. AcrE in E. coli or MexB in P. aeruginosa); controlling ribosomal functions (e.g. rpsl in E. coli or Erm in S. aureus); controlling cytoplasmic membrane assembly (e.g. penicillin binding proteins (PBP)-encoding genes pbp1-3 in E. coli); controlling metabolism (e.g. genes encoding for dihydrofolate reductase such as dhfr in E. coli); causing drug inactivation (e.g. chloramphenicol acetyltransferase-encoding gene such as cat in E. coli and the β-lactamase gene blaZ in S. aureus); and modulating DNA functions (e.g. DNA gyrase-encoding gene gyrA in S. aureus, DNA topoisomerase-encoding gr1 gene in S. aureus). In fungi, the cell product can be, for example, the product of a gene (or genes) controlling cell wall formation (e.g., FKS1, FKS2, CHS 1, CHS2, PMI, and MNN in Saccharomyces cerevisiae), controlling cytoplasmic membrane functions (e.g., SPT, SYR2, IPC1, and IPT1 in S. cerevisiae), controlling DNA integrity (e.g., TOP1 in Candida albicans), controlling protein synthesis (e.g., EF-2 in S. cerevisiae), and for signalling pathways (e.g., CNA1, CNA2 in S. cerevisiae).

The two strains of cells employed in the method of the present invention are substantially the same except for their sensitivity to a particular growth inhibitor or class of growth inhibitors. More particularly, the two strains exhibit essentially the same degree of growth under the same incubation conditions (e.g., temperature and nutrient medium), except that the growth of the first strain is sensitive to (i.e., growth can be substantially reduced or repressed by) exposure to a certain selective growth inhibitor (or class thereof), whereas the growth of the second strain is substantially unaffected when exposed to the same selective growth inhibitor in the same concentration under the same incubation conditions. Accordingly, the first strain of cells is alternatively referred to herein as the “sensitive strain”, and the second strain of cells is alternatively referred to as the “resistant strain”. In this context, the term “essentially the same degree of growth” means that the number of cells of the first strain and the number of cells of the second strain obtained after growth under the same conditions and for the same time differ by no more than about 20% and preferably by no more than about 10%. The term “substantially reduced or repressed” with respect to the sensitivity of the first strain means that there is no visually observable growth of the first strain in the presence of the selective growth inhibitor, whereas visually observable growth of the strain is observed in the absence of the inhibitor under the same growth conditions. The term “substantially unaffected” means that the number of cells of the second strain obtained as a result of growth in the presence of the selective growth inhibitor differs by no more than about 20% and preferably by no more than about 10% from the number of cells obtained as a result of growth of the second strain under the same conditions and for the same time, but in the absence of the inhibitor.

The first and second strains employed in the present invention are generally the same species, and preferably have the same genotype except for their sensitivity to a certain selective growth inhibitor or a certain class of selective growth inhibitors. In one embodiment, the two strains are genetically identical except that the first strain contains a gene which expresses a product essential to cell growth or survival that is sensitive to an inhibitor that acts by selectively inhibiting the function of the product, and the second strain contains a corresponding gene whose expression product is either less sensitive or insensitive to the inhibitor. In another embodiment, the two strains are genetically identical, except that the second strain contains a plasmid that confers resistance to the selective growth inhibitor. In still another embodiment, the two strains are genetically identical, except that the first strain contains a plasmid that confers sensitivity to the selective growth inhibitor.

Pairs of sensitive and resistant strains of cells suitable for use in the present invention are either available to the public or can be generated using methods well known in the art. These pairs are typically (1) a mutated, sensitive strain and the wild type parent strain, (2) a comparatively sensitive wild type strain and a resistant mutant thereof, or (3) a weak, sensitive mutant of a wild type strain and a stronger, resistant mutant thereof. Examples of drug resistant S. aureus strains available through the American Type Culture Collection (ATCC) are: ATCC 27659 (resistant to tetracycline, novobiocin), ATCC 27660 (resistant to tetracycline and streptomycin; inducible resistance to macrolides), and ATCC 33591 (methicillin resistant). Examples of drug-resistant E. coli strains available through the ATCC are ATCC 25252 (resistant to streptomycin) and ATCC 29181 (resistant to trimethoprim and streptomycin). Examples of drug-resistant mycobacterium tuberculosis strains available through the ATCC are ATCC 35820 (resistant to streptomycin) and ATCC 35827 (resistant to kanamycin). All of the foregoing strains can be paired with their respective wild type counterparts from which they were isolated for use in the present invention, wherein the wild type strain would serve as the sensitive strain.

Resistant strains most commonly originate in and are isolated from the clinical environment. Various mechanisms leading to antibiotic resistance are known in the art, including, but not limited to: (1) mutational changes to the original target—where the target is a gene product—leading to, for example, decreased affinity for the drug, (2) up-regulation of molecular pumps or transporters that expel drugs, (3) alterations to the cell wall leading to the reduced entry of drugs, and (4) the acquisition or up-regulation of enzymes that can degrade the drug. Further discussion on antibiotic resistance mechanisms is provided in Davies, Science 1994, 264: 375-382; Walsh, Nature 2000, 406: 775-781; and. Hughes, Nat. Rev. Genet. 2003, 4: 432441.

Resistant strains can also be generated from sensitive strains by growing successive generations of the sensitive strain in vitro in a medium supplemented with incremental amounts of the growth inhibitor of interest to obtain a mutant strain resistant to the inhibitor. The following is a suitable protocol for generating a resistant strain: A drug gradient with small incremental changes can be generated on the surface of an agar plate using a Spiral plater (Spiral Biotech, Norwood, Mass.) (see Hill et al., Rev Infect Dis. 1990, 12 Suppl. 2: S200-209; Wexler et al., J. Clin. Microbiol. 1996, 34: 170-174). Accordingly, a pre-determined volume of liquid drug sample is deposited at the center of the surface of a 100 mm-diameter Petri dish containing 25 ml of solidified medium/agar. The Spiral system is used to distribute the drug in a decreasing, exponential, 1000-fold gradient towards the outer edge. The drug is allowed to briefly soak into the surface of the agar. A swab dipped into a microbial culture containing about 1×10⁹ cells/mL is swiped from the outer surface of the agar toward the center of the petri dish. The plates are then incubated at about 37 ⁰C for about 24 to about 48 hours. The edge of growth within the bacterial streak that is closest to the center and to the highest concentration of drug is sampled and grown in liquid broth overnight. A swab from this overnight culture is then used to treat a freshly prepared gradient plate. This technique allows for successive rounds resulting in cells with ever increasing levels of resistance. After multiple passages resistant colonies can be isolated in which the MIC has increased from 8-fold to 32-fold.

Strains resistant to selective growth inhibitors can also be genetically engineered using recombinant technology well known in the art. For example, a plasmid harboring a drug-resistance gene can be introduced into a bacterial strain and render it resistant to a drug. The methodology for transferring plasmids into bacteria is well known (see, e.g., Ausubel et al., Current Protocols in Molecular Biology, Wiley & Sons, New York, 1998). A specific example involves the gene encoding for chloramphenicol acetyl transferase, which inactivates chloramphenicol (Shaw et al., Annu. Rev. Biophys. Biophys. Chem. 1991, 20: 363-86).

As noted above, strains more sensitive to a class of drugs than the parent wild type can also be obtained. For the purposes of this invention, the new strain so obtained is the “sensitive” strain, and the wild type is the “resistant” strain. Strains of bacteria can be made more sensitive to certain drugs by means of down-regulating the gene encoding the target of that drug. Methods for engineering such strains are described, for example, in Forsyth et al., Mol. Microbiol. 2000, 43:1387-1400; Ji et al., Science 2001, 293: 2266-2269;); and DeVito et al., Nat. Biotechnol. 2002, 20: 478483).

Similar strategies can be applied to the procurement and/or preparation of resistant-sensitive pairs of eukaryotic cells (e.g., fungal strains for screening substances for antifungal activity).

As is clear from the above description, a positive result for the method of the present invention (i.e., a zone of no growth punctuated with colonies of the resistant strain in the treated, incubated nutrient medium, indicating that the test substance is a selective growth inhibitor) is the consequence of the preferential growth of relatively few resistant colonies. There is a circumstance, believed to be uncommon, where visible colonies might not arise solely from the co-seeded resistant cells. This circumstance will arise when the strain of sensitive cells is sensitized to a given class of drugs by virtue of the presence of a plasmid that is relatively unstable, such that spontaneous loss of the plasmid (e.g. a 1% loss rate over 2-4 generations) will generate revertant cells which are more resistant than the parent cells. The revertant cells can then grow into detectable resistant colonies. This circumstance can arise, for example, when the plasmid expresses an antisense RNA molecule which in turn causes the bacterium in which it resides to be more sensitive to drugs affecting the biological pathway in which the gene corresponding to the antisense is involved. Further description of such spontaneously-occuring resistant colonies can be found in WO 2004/009835. This circumstance will typically not result in a false positive in the method of the present invention, because visually observable colonies originating from both the co-seeded resistant strain and the spontaneous (revertant) strain should arise concurrently.

A false positive can be generated in the method of the present invention in the circumstance where cells of the sensitive strain possess an uncommonly rapid drug resistance mechanism that leads to the appearance of resistant daughter cells. One example is the rapid loss in a bacterial strain (e.g., E. coli or S. aureus) of a glycerophosphate transport system through which fosfomycin enters the bacteria, leading to fosfomycin resistance (see Dulaney et al., J. Antibiotics (Tokyo) 1977, 30: 252-261). Fosfomycin (or another test substance that enters the bacteria via the rapidly lost glycerophosphate transport system) could therefore generate a false positive in the method of the present invention; i.e., the assay result would lead to the false conclusion that the fosfomycin acts at the target by which the sensitive and resistant strains differ (unless of course the co-dosed resistant strain is itself resistant to fosfomycin). This type of false positive is believed to be rare; i.e., no example other than the just-described fosfomycin case is known to the inventor. If suspected, this type of false positive can be detected by testing the substance(s) against the sensitive strain paired with two or more resistant strains each with a different mechanism of action. If all of the tests give a positive result, then it is a false positive. If only one assay is performed, the false positive can be identified in a subsequent test or screen. For example, all of the positives can be tested against the sensitive strain alone, in which case only a false-positive like fosfomycin would generate visible colonies.

The following examples serve only to illustrate the invention and its practice. The examples are not to be construed as limitations on the scope or spirit of the invention. In the examples:

MRSA is a methicillin-resistant, Staphylococcus aureus strain, which is an “R35” clinical isolate obtained from Dr. J. Wolfson at the Harvard Medical School. Equivalent MRSA strains available through the ATCC include numbers 700689 and BAA-37 to BAA44. Use and drug susceptibilities of this strain have been described in the following works: Rosen et al., Science 1999, 283:703-706; Hicks et al., Clin. Microbiol. Infect. 2002, 8(11): 753-7; Pelak et al., J. Chemother. 2002, 14(3): 227-33; and Pelak et al., Diagn. Microbiol. Infect. Dis. 2002, 43(2): 129-33.

MSSA is a methicillin-sensitive S. aureus strain from Merck stocks. Equivalent MSSA strains available through the ATCC include numbers 25923 and 29213. The properties of the instant MSSA strain are the same as those set forth in Rosen et al., Science 1999, 283:703-706

CRMRSA is a chloramphenicol-resistant MRSA strain obtained via the Spiral plater technique set forth above.

DH5 is an ampicillin-sensitive Escherichia coli strain, which is described in D. Hanahan, J. Mol. Biol. 1983, 166: 557-580.

pKS-DH5 is a plasmid-bearing version of the DH5 strain wherein the plasmid pKS (see Short et al., Nucleic Acids Res. 1988, 16: 7583-7600) confers ampicillin resistance.

ATCC 9637 is a streptomycin-sensitive wild-type E. coli strain.

MB941 is a streptomycin-resistant E. coli strain which can be obtained by the Spiral plater technique set forth above.

S-eEF2 is a Saccharomyces cerevisiae strain (yeast strain) that expresses the EF2 gene and is sensitive to sordarin. S-eEF2 is described in Justice et al., J. Biol. Chem. 1998, 273: 3148-3151.

HS-eEF2 is an S. cerevisiae human eEF2 chimera strain that expresses a yeast-human EF2 chimera gene and is insensitive to sordarin. HS-eEF2 is described in Shastry et al., Microbiology 2001, 147: 383-390.

All test substances were obtained from Sigma Chemicals, with the exception of linezolid which was obtained from Pharmacia Inc., and sordarin which was prepared in the manner described in Justice et al., J. Biol. Chem. 1998, 273: 3148-3151.

### EXAMPLE 1

### Identification of Antibacterial Agents

Assay Protocol A

Each of a pair of bacterial strains, one sensitive and one resistant to a particular antibacterial agent, was grown by inoculating 25 mL of LB broth (i.e., 10 g of tryptone, 5 g of yeast extract, and 10 g NaCl per liter of water) with 1 to 5 colonies in an Erlenmeyer flask, and then holding the mixture overnight at a temperature of 37° C. while agitating the mixture via rotary shaking (200 rpm). Each batch was allowed to cool to room temperature (about 25° C.). Following OD measurements at λ=600 nm to ensure that both strains have grown to similar densities (i.e., a variation of 10% or less), a sample from the batch of the “sensitive” strain was diluted 10-fold in LB growth medium, and a sample of the “resistant” strain was diluted 500-fold or more to provide a final ratio (i.e., co-seeding ratio) of 50 or more sensitive cells to 1 resistant cell. A 750 μL sample of each of the diluted strains was then mixed into 20 mL of molten agar/growth medium agar (melt temperature=48° C.) (KD Medical's Cat. No. BLF-7070) and poured into rectangular 8.5×12.5 cm Omnitray dishes (Nalge NUNC Intl. Cat No. 242811). Wells were cast by applying custom-made pins (about 3 mm in diameter and 5 mm deep; Nalge NUNC Intl.) into the surface of the molten mixture (48 or 96 pins arrayed over the 8.5×12.5 cm surface) and then allowing the mixture to harden to provide an assay plate. Test samples dissolved in DMSO were added to the wells, after which the assay plate was incubated at 37° C. for a time sufficient to allow visual detection of cell colonies (i.e., 12 to 18 hours). The plates were then examined for zones of growth and inhibition under ambient light.

Assay Results

The tests set forth in Table 1 were conducted in the manner described in Assay Protocol A, except that in the case of the streptomycin-resistant strain, the inoculated liquid growth medium also contained 25 ug/mL of streptomycin in order to ensure that resistance was not lost during growth.

TABLE 1

| | | | Co- | | |
|---|---|---|---|---|---|
| Run | Sensitive | Resistant | Seeding | Test substance¹ | |
| No. | Strain | Strain | Ratio | (concentration) | Result |
| 1 | MSSA | MRSA | 500:1 | chloramphenicol | |
| | | | | (100 μg/mL) | |
| 2 | MSSA | MRSA | 500:1 | streptomycin | |
| | | | | (1 mg/mL) | |
| 3 | MSSA | MRSA | 500:1 | cefoxitin | |
| | | | | (500 μg/mL) | |
| 4 | MRSA | CRMRSA | 100:1 | ciprofloxacin | |
| | | | | (50 μg/mL) | |
| 5 | MRSA | CRMRSA | 100:1 | chloramphenicol | |
| | | | | (1 mg/mL) | |
| 6 | DH5 | pKS-DH5 | 500:1 | ampicillin | |
| | | | | (100 mg/mL) | |
| 7 | ATCC 9637 | MB941 | 1000:1 | tetracyclin | |
| | | | | (1 mg/mL) | |
| 8 | ATCC 9637 | MB941 | 1000:1 | streptomycin | |
| | | | | (500 μg/mL) | |

¹In all cases 10 μL of a solution of the test substance in DMSO was added to the well of the test plate.

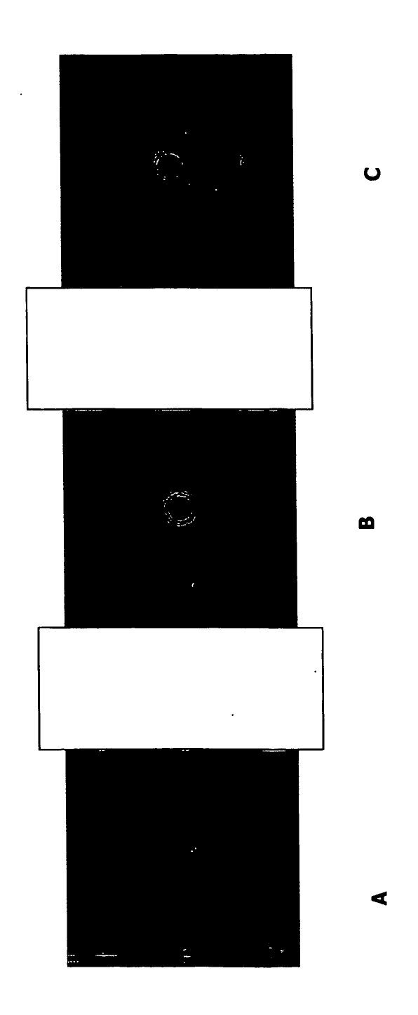

Figures 1A, 1B, and 1C each show a zone of growth inhibition in the presence of chloramphenicol, streptomycin, and cefoxitin respectively. The inhibition zone in FIG. 1A visually contains no resistant colonies, the 1B zone contains a few colonies at or near the border of the inhibition zone, and the 1C zone contains a multitude of resistant colonies throughout the inhibition zone. The partial positive response seen with streptomycin in 1B indicates that the MRSA strain displays resistance to a broad spectrum of antibiotics including some that do not act like methicillin. The multiple resistance of MRSA strains has been disclosed, for example, in Chambers, Clin. Microbiol. Rev. 1997, 10: 781-791 and in Crisóstomo et al., PNAS 2001, 98: 9865-9870. The full positive response exhibited by cefoxitin in 1C indicates that this antibiotic has the same mode of action as methicillin; i.e., it is a cell wall inhibitor (see, e.g., Farrar Jr. et al., J. Infect. Dis. 1976, 133: 691-695).

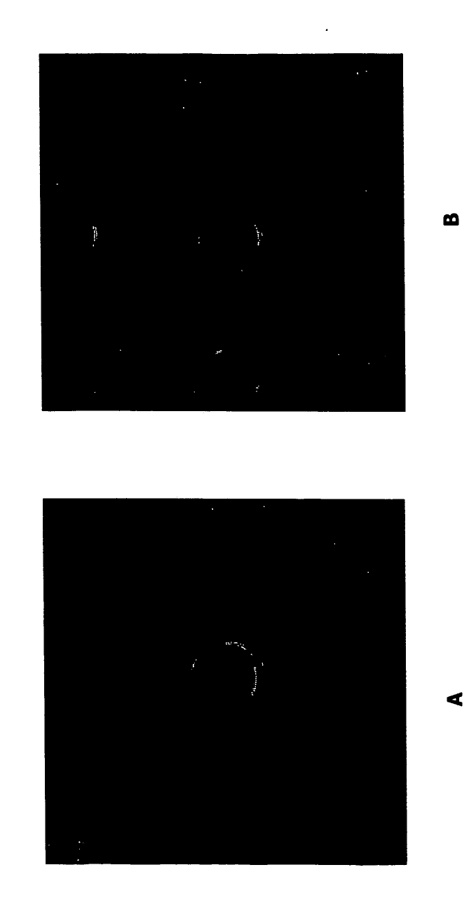

FIG. 2A shows a clear zone of no growth, but FIG. 2B shows a zone of no growth containing isolated colonies of resistant bacteria. The results set forth in FIGS. 2A and 2B indicate that ciproflaxin does not have the same mode of action as chloramphenicol, which is known to act against protein translation (see, e.g., Pestka, Ann. Rev. Microbiol. 1971, 25: 487-562).

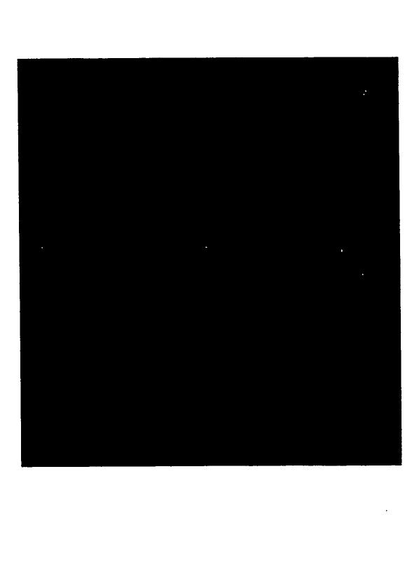

The results shown in FIG. 3 (i.e., isolated resistant colonies within a zone that otherwise exhibits no growth) confirm the resistance of pKS-DH5 to ampicillin, which is a cell wall inhibitor (see, e.g., Sykes et al., Pharmacol. Ther. 1985, 29: 321-352.

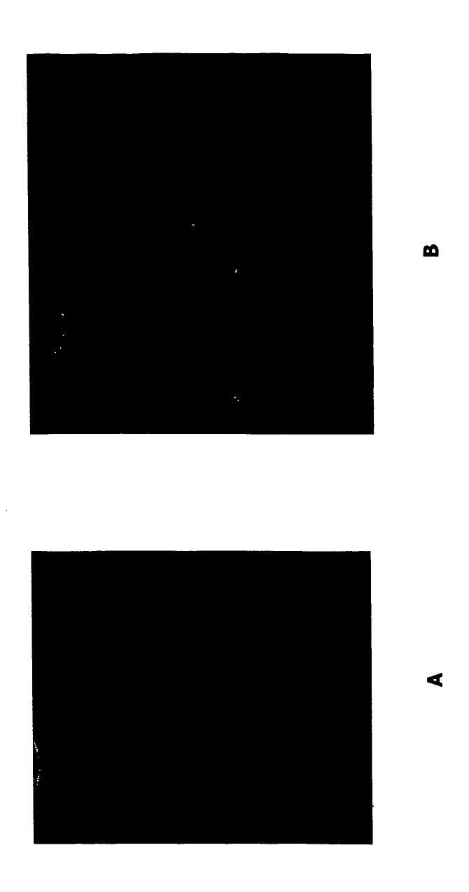

FIG. 4A shows a clear zone of no growth, but FIG. 4B shows a zone of no growth containing isolated colonies of resistant bacteria. The results shown in FIGS. 4A and 4B indicate that tetracylin, a known antibacterial, has a mode of action different from that of streptomycin. This result is consistent with the art, wherein it is known that streptomycin acts against the ribosome at the initial selection and at the proof-reading step and that tetracyclin acts on the ribosome by blocking the binding of aminoacylated tRNA to the A site (see, e.g., Bilgin et al., J. Mol. Biol. 1994, 235: 813-824; Geigenmuller et al., Eur. J. Biochem. 1986, 161: 723-726; Karimi et al., Eur. J. Biochem. 1994, 226:, 355-360; Karimi et al., EMBO J. 1996, 15: 1149-1154; Maxwell, Biochim. Biophys. Acta 1967, 138: 337-346; and Ruusala et al., Mol. Gen. Genet. 1984, 198: 100-104).

### EXAMPLE 2

### Identification of Antifungal Agents

Assay Protocol B The S. cerevisiae yeast strains, S-eEF2 and HS-eEF2, were each grown overnight from a single colony in 5 mL YPAD (i.e., 1% yeast extract, 2% peptone, 0.004% adenine sulfate, and 2% glucose) in an Erlenmeyer flask at 29° C. with shaking on a roller drum. The sensitive strain S-eEF2 was diluted with additional YPAD to give a final absorbance at λ=600 nm of 0.01 (control =YPAD), and the resistant strain HS-eEF2 was diluted to an absorbance at λ=600 nm of 0.0002. Each strain (5 mL) was added to a glass test tube containing 25 mL of molten YPAD agar equilibrated at 50° C. The mixture was then stirred briefly and poured onto sterile disposable 86×128-mm plastic dishes (NUNC Cat. No. 242811). The dishes were cooled for 5 minutes at room temperature, dried in a hood, and then stored for no more than 2 days until ready for treatment with test substances. Test substances were dissolved in the appropriate solvent and a suitable portion of the solution was spotted on the co-seeded plates. The plates were incubated overnight at 29° C., and then inspected visually for the appearance of zones of growth and inhibition, and particularly for the appearance of resistant colonies capable of growing in the presence of the test substances.

Further description of procedures and media suitable for growth and genetic manipulation of yeast strains can be found in Sherman et al., Laboratory Course Manual for Methods in Yeast Genetics, Cold Spring Harbor Laboratory, Cold Spring Harbor N.Y., 1986.

Assay Results

The tests set forth in Table 2 were conducted in the manner described in Assay Protocol B.

TABLE 2

| Run | Sensitive | Resistant | Co-Seeding | Test substance¹ | |
|---|---|---|---|---|---|
| No. | Strain | Strain | Ratio | (amount) | Result |
| 9 | S-eEF2 | HS-eEF2 | 50:1 | enfumafungin | |
| | | | | (0.2 μg) | |
| 10 | S-eEF2 | HS-eEF2 | 50:1 | sordarin | |
| | | | | (2 μg) | |

¹A solution of the test substance in DMSO is employed.

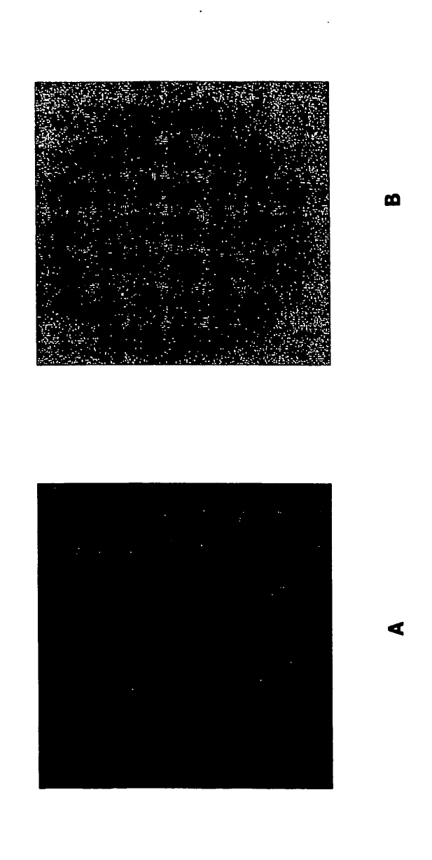

As noted earlier, HS-eEF2 is resistant to antifungal agents which act by inhibiting the function of elongation factor 2 (EF2). FIG. 5A shows a visually clear zone exhibiting no growth of either the sensitive or the resistant strain in the presence of enfumafungin. Accordingly, the assay shows that enfumafungin is an effective growth inhibitor, but that it does not act against EF2. On the other hand, FIG. 5B shows a zone of growth inhibition in which resistant colonies are present. Accordingly, the assay shows that sordarin is a growth inhibitor that acts against EF2.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A method for identifying selective growth inhibitors, which comprises:
(A) providing a semi-solid nutrient medium co-seeded with a first and a second strain of cells; wherein (i) the two strains are substantially the same except that the growth of the first strain is sensitive to exposure to an inhibitor that acts by selectively inhibiting the function of a product of the cell required for its growth or survival, and the growth of the second strain is substantially resistant to exposure to the selective inhibitor, and (ii) first strain is present in the nutrient medium in an amount substantially in excess of the second strain;
(B) treating the nutrient medium with a test substance;
(C) incubating the treated medium under conditions suitable for growth of the two strains of cells; and
(D) examining the incubated nutrient medium for growth inhibition; wherein:
(1) if the incubated nutrient medium exhibits a clear zone indicative of essentially no growth due to the death of all or substantially all of the cells, then the test substance is or contains a growth inhibitor that does not selectively inhibit the targeted cell product;
(2) if the incubated nutrient medium does not exhibit a zone of no growth due to the survival and growth of all or substantially all of the cells, then the test substance is not or does not contain a growth inhibitor; and
(3) if the incubated nutrient medium exhibits a zone of no growth except for one or more small cell colonies in the zone due to the survival and growth of the second strain, then the test substance is or contains a growth inhibitor that selectively inhibits the targeted cell product.

2. The method according to claim 1, wherein the semi-solid nutrient medium is an agar plate or an agarose plate.

3. The method according to claim 1, wherein treating in Step B comprises applying the test substance to the surface of the semi-solid nutrient medium.

4. The method according to claim 1, wherein the incubation in Step C is conducted at a temperature in a range of from about 20° C. to about 45° C.

5. The method according to claim 4, wherein the incubation in Step C is conducted for a time in a range of from about 8 to about 120 hours.

6. The method according to claim 1, wherein the first strain and the second strain are co-seeded in the semi-solid nutrient medium in a ratio of at least about 25 cells of the first strain per cell of the second strain.

7. The method according to claim 6, wherein the co-seeding ratio is at least about 100 cells of the first strain per cell of the second strain.

8. The method according to claim 1, wherein in Step B, the nutrient medium is treated with a plurality of test substances, with each test substance being applied to a distinct portion of the surface of the nutrient medium; and wherein in Step D the nutrient medium is examined for growth inhibition by each of the plurality of test substances by examining each of the corresponding distinct portions of the nutrient surface.

9. The method according to claim 1, wherein the growth of the first strain is repressed by exposure to an inhibitor that acts by selectively inhibiting the function of a specific gene product of the cell required for the cell's growth or survival, and the growth of the second strain is substantially resistant to exposure to the selective inhibitor.

10. The method according to claim 1, wherein the first and second strains are cells are either bacterial strains or fungal strains.

11. The method according to claim 10, wherein the first and second strains of cells are bacterial strains and the test substance is being tested to determine whether it is an antibacterial agent that acts by selectively inhibiting the function of a cell product required for the growth or survival of the bacteria.

12. The method according to claim 11, wherein the bacterial strains are selected from the group consisting of Staphylococcus, Streptococcus, Enterococcus, Escherichia, Klebsiella, Haemophilus, Enterobacter, Acinetobacter, Bacillus, Stenotrophomonas, Burkholderia, Salmonella, and Pseudomonas.

13. The method according to claim 12, wherein the bacterial strains are selected from the group consisting of Streptococcus pneumonia, Staphylococcus aureus, Enterococcus faecalis, Enterococcus faecium, Escherichia coli, Klebsiella pneumoniae, Haemophilus influenzae, Enterobacter cloacae, Pseudomonas aeruginosa, Acinetobacter baumanii, Bacillus subtilis, Stenotrophomonas maltophilia, Salmonella typhimurium, and Burkholderia cepacia.

14. The method according to claim 10, wherein the first and second strains of cells are fungal strains and the test substance is being tested to determine whether it is an antifungal agent that acts by selectively inhibiting the function of a cell product required for the growth or survival of the fungi.

15. The method according to claim 14, wherein the fungal strains are selected from the group consisting of Cryptococcus spp., Candida spp., Aspergillus spp., Histoplasma spp., Coccidioides spp., Paracoccidioides spp., Blastomyces spp., Fusarium spp., Sporothrix spp., Trichosporon spp., Scedosporium, Rhizopus spp., Pseudallescheria spp., dermatophytes, Paeciliomyces spp., Alternaria spp., Curvularia spp., Exophiala spp., Schizosaccharomyces spp., Wangiella spp., Dematiaceous fungi, Pneumocystis spp, Absidia spp., Blastocystis spp., and Epidermophyton spp.

16. The method according to claim 15, wherein the fungal strains are selected from the group consisting of Saccharomyces cerevisiae, Aspergillus nidulans, Aspergillus fumigatus, Cryptococcus neoformans, Coccidioides immitis, Schizosaccharomyces pombe, Pneumocystis carinii, Candida albicans, Absidia corymbifera, Aspergillus flavus, Blastocystis hominis, Blastomyces dermatitidis, Coccidioides immitis, Cryptococcus neoformans, Epidermophyton floccosum, Sporothrix schenckii, and Trichophyton rubrum.

17. A method for identifying selective growth inhibitors, which comprises:
(A) providing an agar plate or an agarose plate, the plate co-seeded with a first and a second strain of cells in a ratio of at least about 25 cells of the first strain per cell of the second strain; wherein the two strains are substantially the same except that the growth of the first strain is sensitive to exposure to an inhibitor that acts by selectively inhibiting the function of a product of the cell required for its growth or survival, and the growth of the second strain is resistant to exposure to the selective inhibitor;
(B) treating the plate with a test substance by applying the test substance to the surface of the plate;
(C) incubating the treated plate at a temperature in a range of from about 20° C. to about 45° C. and for a time in a range of from about 4 to about 96 hours; and
(D) examining the incubated plate for growth inhibition; wherein:
(1) if the incubated plate exhibits a clear zone indicative of essentially no growth due to the death of all or substantially all of the cells, then the test substance is or contains a growth inhibitor that does not selectively inhibit the targeted cell product;
(2) if the incubated plate does not exhibit a zone of no growth due to the survival and growth of all or substantially all of the cells, then the test substance is not or does not contain a growth inhibitor; and
(3) if the incubated plate exhibits a zone of no growth except for one or more small cell colonies in the zone due to the survival and growth of the second strain, then the test substance is or contains a selective growth inhibitor that selectively inhibits the targeted cell product.

18. The method according to claim 17, wherein the first and second strains are co-seeded in the plate in a ratio of at least about 100 cells of the first strain per cell of the second strain.

19. The method according to claim 18, wherein the first and second strains are co-seeded in the plate in a ratio in a range of from about 25 to about 1000 cells of the first strain per cell of the second strain.

20. The method according to claim 17, wherein the first and second strains are bacterial strains.
